(19) United States
(12) Patent Application Publication
(10) Pub. No.: US 2025/0078999 A1
(43) Pub. Date: Mar. 6, 2025

(54) INFORMATION PROCESSING DEVICE, INFORMATION PROCESSING METHOD, AND PROGRAM

(71) Applicant: NEC Corporation, Minato- ku, Tokyo (JP)

(72) Inventors: Masahiro HAYASHITANI, Tokyo (JP); Kosuke NISHIHARA, Tokyo (JP); Eiji YUMOTO, Tokyo (JP)

(73) Assignee: NEC Corporation, Minato- ku, Tokyo (JP)

(21) Appl. No.: 18/288,866

(22) PCT Filed: Jan. 12, 2023
(86) PCT No.: PCT/JP2023/000649

§ 371 Date: Oct. 30, 2023

(51) Int. Cl. G16H 50/20; G16H 40/20

(52) U.S. Cl. CPC G16H 50/20; G16H 40/20

(57) ABSTRACT

An information processing device 100 of the present disclosure includes a data acquisition unit 121 and a data selection unit 122. The data acquisition unit 121 acquires biological data measured from a person whose state related to agitation is to be determined, and a determination result of the state by a determiner with respect to the person. The data selection unit 122 selects learning data from the biological data on the basis of a skill value and the determination result. The skill value represents the ability of determining the state and is set for the determiner. Selection of the learning data is optimized by the data selection unit 122 of the present disclosure.

### TECHNICAL FIELD

The present disclosure relates to an information processing device, an information processing method, and a program.

### BACKGROUND ART

In a hospital, the state of a patient is monitored and, for example, detection of an agitated state is performed. An agitated state means a state in which the patient is very cautious to the surroundings, uncalm, and excited. Patent Literature 1 discloses an example of a method of detecting an agitated state. Patent Literature 1 describes generating a model by learning a relationship between biological information measured from a patient in the past and an agitated state or a non-agitated state of the patient, and detecting an agitated state based on such a model.

### CITATION LIST

### Patent Literature

Patent Literature 1: WO 2020/161901 A

### SUMMARY OF INVENTION

### Technical Problem

However, it is difficult to determine whether or not a patient is in an agitated state. Therefore, it is difficult to identify whether or not the biological information measured from a patient is one in an agitated state, so that it is impossible to improve the quality of biological information to be learned. As a result, there is a problem that it is impossible to improve the accuracy of detecting an agitated state using a model generated through learning of biological information.

Therefore, an object of the present invention is to provide an information processing device that can solve the above-described problem, that is, a problem that it is impossible to improve the accuracy of detecting an agitated state of a person.

### Solution to Problem

An information processing device, according to one aspect of the present disclosure, is configured to include
a data acquisition unit that acquires biological data measured from a person whose state related to agitation is to be determined, and a determination result of the state by a determiner with respect to the person, and
a data selection unit that selects learning data from the biological data on the basis of a skill value and the determination result. The skill value represents the ability of determining the state and is set for the determiner.

Further, an information processing method, according to one aspect of the present disclosure, is configured to include
acquiring biological data measured from a person whose state related to agitation is to be determined, and a determination result of the state by a determiner with respect to the person, and
selecting learning data from the biological data on the basis of a skill value and the determination result. The skill value represents the ability of determining the state and is set for the determiner.

A program, according to one aspect of the present disclosure, is configured to cause a computer to execute processing to
acquire biological data measured from a person whose state related to agitation is to be determined, and a determination result of the state by a determiner with respect to the person, and
select learning data from the biological data on the basis of a skill value and the determination result. The skill value represents the ability of determining the state and is set for the determiner.

### Advantageous Effects of Invention

With the configurations described above, the present disclosure can improve the accuracy of detecting an agitated state of a person.

### BRIEF DESCRIPTION OF DRAWINGS

FIG. 1 is a block diagram illustrating a configuration of an information processing device according to a first example embodiment of the present disclosure.

FIG. 2 illustrates a state of processing by the information processing device disclosed in FIG. 1.

FIG. 3 illustrates a state of processing by the information processing device disclosed in FIG. 1.

FIG. 4 illustrates a state of processing by the information processing device disclosed in FIG. 1.

FIG. 5 is a flowchart illustrating an operation of the information processing device disclosed in FIG. 1.

FIG. 6 is a flowchart illustrating an operation of the information processing device disclosed in FIG. 1.

FIG. 7 is a block diagram illustrating a hardware configuration of an information processing device according to a second example embodiment of the present disclosure.

FIG. 8 is a block diagram illustrating a configuration of the information processing device according to the second example embodiment of the present disclosure.

### DESCRIPTION OF EMBODIMENTS

### First Example Embodiment

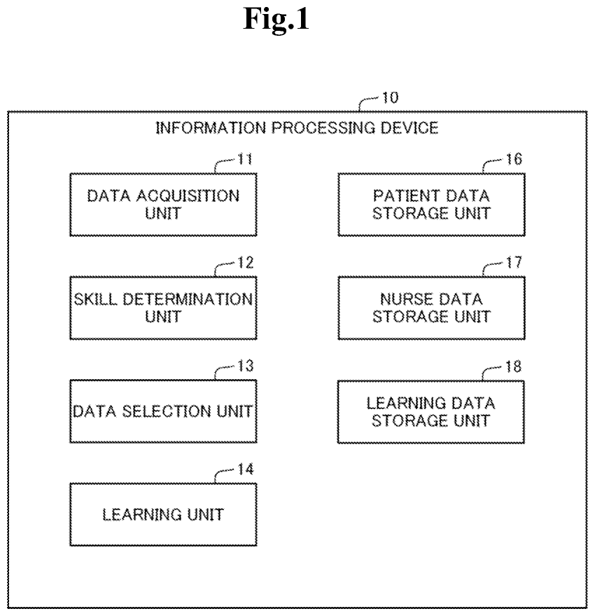

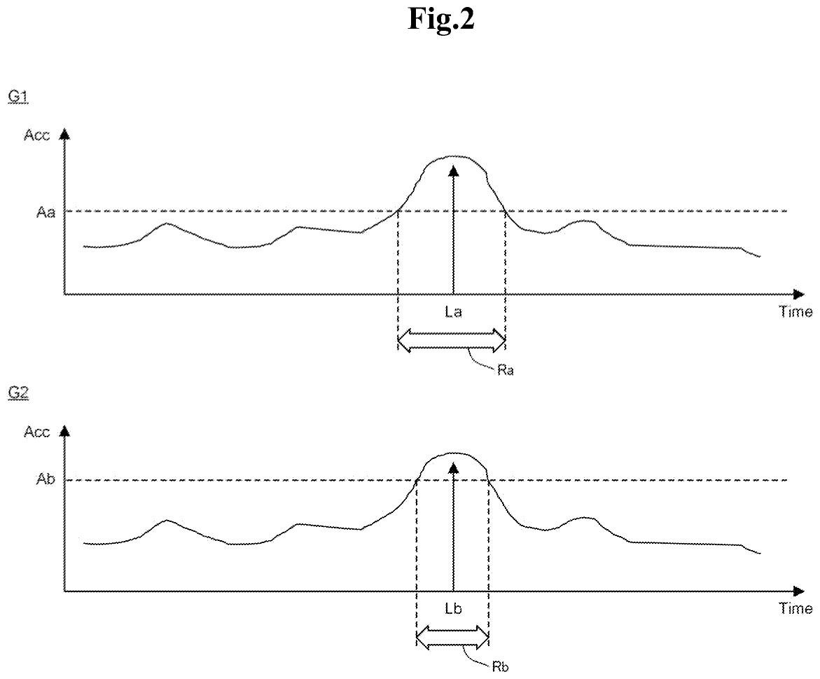

A first example embodiment of the present disclosure will be described with reference to FIGS. 1 to 6. FIG. 1 is a diagram for explaining a configuration of an information processing device, and FIGS. 2 to 6 are diagrams for explaining a processing operation of the information processing device.

### Configuration

An information processing device 10 of the present embodiment has a function of generating a model for detecting an agitated state of a patient in the hospital. In particular, the information processing device 10 has a function of acquiring biological data measured from a patient, and select learning data to be used for generating a model by machine learning, as described below.

Specifically, the information processing device 10 is configured of one or a plurality of information processing devices each having an arithmetic unit and a storage device. As illustrated in FIG. 1, the information processing device 10 includes a data acquisition unit 11, a skill determination unit 12, a data selection unit 13, and a learning unit 14. The functions of the data acquisition unit 11, the skill determination unit 12, the data selection unit 13, and the learning unit 14 can be realized through execution, by the arithmetic unit, of a program for realizing the respective functions stored in the storage device. The information processing device 10 also includes a patient data storage unit 16, a nurse data storage unit 17, and a learning data storage unit 18. The patient data storage unit 16, the nurse data storage unit 17, and the learning data storage unit 18 are configured of storage devices. Hereinafter, the respective constituent elements will be described in detail.

The data acquisition unit 11 acquires biological data measured from a patient (person) whose state related to agitation such as an agitated state or a calm state is to be determined. The biological data is data representing a motion and a state of a patient measured by a sensor attached to the patient. In the present embodiment, the biological data is acquired by measuring acceleration. The data acquisition unit 11 stores the acquired biological data in association with the time at which the data is measured, in the patient data storage unit 16. That is, as illustrated in FIG. 2, the data acquisition unit 11 acquires biological data represented as time-series data on the graph in which the horizontal axis shows the time (Time) and the vertical axis shows the acceleration (Acc). However, the biological data is not limited to acceleration of the patient. It may be another type of biological data representing the motion or state of the patient such as heartbeat interval or skin temperature.

The data acquisition unit 11 also acquires imaging data in which the motion of a patient when the biological data is measured is imaged. It is assumed that the time of imaging data is in synchronization with the time of the biological data. The data acquisition unit 11 stores the acquired imaging data in association with the biological data of the same patient, in the patient data storage unit 16.

The data acquisition unit 11 also acquires a result of determining the state by a nurse (determiner) with respect to the patient. In the present embodiment, it is assumed that a nurse determines the level of agitation in stages by reviewing the imaging data stored in the patient data storage unit 16, such as the patient “is in an agitated state” or “may be in an agitated state”. Note that “being in an agitated state” has a higher level of agitation than “may be in an agitated state”. Then, the data acquisition unit 11 stores, in the patient data storage unit 16, information identifying the nurse who made determination, a label representing the agitation level that is a determination result, and the determination time that is the time of determination on the imaging data, in association with the imaging data of the patient. Note that the determination described above is not limited to be performed by a nurse. It may be performed by any person who is subject to calculation of a skill value representing the ability of determining the state of a patient, as described below.

The data acquisition unit 11 also acquires correct data of a state with respect to the patient. In the present embodiment, correct data is a determination result by a doctor who can determine, from the imaging data of a patient, the agitation level such as “being in an agitated state” or “may be in an agitated state” with high accuracy, or by a nurse having high determination ability. Then, the data acquisition unit 11 stores, in the patient data storage unit 16, a label representing the agitation level that is determined to be correct data, and the determination time that is the time of determination on the imaging data, in association with the imaging data of the patient. Note that the correct data described above is not limited to a determination result by a doctor or a nurse having high determination ability. A determination result by any person may be used, and a determination result by an analysis result using a computer may be used.

In the above description, in the determination by a nurse and correct data, the agitation level of a patient is expressed in two stages, that is, “being in an agitated state” and “may be in an agitated state”. However, the agitation level may be one, that is, “being in an agitated state”, or may be expressed in more stages. For example, the agitation level of a patient may be expressed based on a predetermined sedation scale called Richmond Agitation-Sedation Scale (RASS). Moreover, in the determination by a nurse and correct data, “being calm” or “calm level” may be used as a state of a patient.

The skill determination unit 12 determines a skill value representing the determination ability of a nurse who determined the state of a patient as described above. Specifically, the skill determination unit 12 first reads a label indicating the agitation level that is a determination unit with respect to a given patient by a nurse who is a subject to skill determination, and a label that is correct data with respect to the same patient, stored in the patient data storage unit 16. At that time, with respect to the nurse who is subject to skill determination, the skill determination unit 12 reads determination results and correct data for a plurality of patients. Then, the skill determination unit 12 compares a label of a determination result by the nurse with a label of the correct data, and calculates the score according to the difference. At that time, the skill determination unit 12 calculates the score in such a manner that the value of the score becomes higher as the number of cases where a label of a determination result by the nurse and a label of correct data match is larger, or that the value of the score becomes higher as the determination time at which a label of a determination result and a label of correct data match is nearer. Then, the skill determination unit 12 checks whether the calculated value of the score is equal to or larger than a predetermined threshold, and when it is equal to or larger than the threshold, the skill value of the nurse is determined to be high skill, while when it is less than the threshold, the skill value of the nurse is determined to be low skill. Note that in this example, high skill means that the skill value is higher than low skill. That is, the skill determination unit 12 determines that the skill value becomes higher as the difference between the determination result by the nurse and the correct data is smaller.

Note that while two values, that is, high skill and low skill, are set as skill values of a nurse in the above description, skill values may be set in stages using more values. For example, it is possible to set numerical values in five stages such as “1, 2, 3, 4, and 5” in which the skill value is higher as the numerical value is larger, and determine the skill value to be any value according to the value of the score described above. However, the skill value may be data of any form as long as it is a value representing the determination ability of a nurse.

Then, the skill determination unit 12 stores the skill value determined for each nurse in the nurse data storage unit 17 in association with the identification information of the nurse. Note that the skill value of the nurse may be determined by another information processing device or by another method and set in advance, and stored in the nurse data storage unit 17. Therefore, the skill determination unit 12 may not be provided in the information processing device 10.

The data selection unit 13 selects learning data to be used for performing machine learning to generate a model, from biological data of patients stored in the patient data storage unit 16. In particular, the data selection unit 13 selects learning data from biological data, by means of a selection method set according to the skill value of the nurse who determined the state of a patient.

Specifically, the data selection unit 13 first searches the biological data of a patient for the determination time at which it is determined as “being in an agitated state” or “may be in an agitated state”, that is, the determination time at which an agitation label is given. Then, with respect to the biological data to which an agitation label is given, the data selection unit 13 sets a time section based on the determination time according to the skill value of the nurse who made determination, and sets the biological data in the set time section as learning data. For example, in the case where there is determination time at which a label of “being in an agitated state” or “may be in an agitated state” is given in the biological data, and where the skill value of the nurse who made determination is “high skill”, the data selection unit 13 sets a time section having a predetermined time width around the determination time, and selects the biological data in such a time section as learning data. Then, the data selection unit 13 stores the biological data selected as learning data, in the learning data storage unit 18 while giving a “agitated state” label thereto.

Here, an example of a data selection method in the case where the skill value of a nurse who made determination is “high skill” will be described with reference to FIG. 2. First, a reference sign G1 on the upper drawing of FIG. 2 indicates a method of selecting learning data from acceleration Acc that is biological data to which a label “being in an agitated state” is given. In that case, the data selection unit 13 sets an acceleration threshold Aa that is a value lower than the acceleration Acc at determination time La to which a label “being in an agitated state” is given, and sets a time section Ra having a predetermined time width in which the acceleration Acc that is equal to or higher than the acceleration threshold A is measured. In this way, when the skill value of the nurse who made determination is “high skill”, the data selection unit 13 selects the acceleration Acc in the time section Ra having the predetermined time width around the determination time La as learning data. Moreover, when the skill value of the nurse who made determination is “high skill”, the data selection unit 13 duplicates the acceleration data in the set time section Ra and selects it as learning data. For example, the data selection unit 13 makes two or more duplicates of the acceleration data in the time section Ra having a predetermined time width, and selects them as learning data. Note that the acceleration threshold Aa may be a value calculated from the acceleration at the determination time La, or may be a value set in advance. For example, the acceleration threshold Aa having a value lower than the acceleration that can be determined as “being in an agitated state” may be set, from analysis and a study result of past biological data of a plurality of persons.

A reference sign G2 in the lower drawing of FIG. 2 indicates a method of selecting learning data from the acceleration Ace that is biological data to which a label “may be in an agitated state” is given. In that case, the data selection unit 13 sets an acceleration threshold Ab that is a value lower than the acceleration Acc at determination time Lb to which a label “may be in an agitated state” is given, and sets a time section Rb having a predetermined time width in which the acceleration Acc that is equal to or higher than the acceleration threshold Ab is measured. In this way, when the skill value of the nurse who made determination is “high skill”, the data selection unit 13 selects the acceleration Acc in the time section Rb having the predetermined time width around the determination time Lb as learning data. At that time, the acceleration threshold Ab is set to be a value higher than the acceleration threshold Aa that is set when a label “being in an agitated state” is given. Therefore, when it is determined as “may be in an agitated state” in which the agitation level is lower than the case that is determined as “being in an agitated state”, the time section Rb around the determination time Lb is selected to be narrower. This means that the time section is set while being changed according to the agitation level determined by the nurse. In particular, the time section is set to be longer as the determined agitation level is higher. Moreover, when the skill value of the nurse who made determination is “high skill”, the data selection unit 13 duplicates the acceleration data in the set time section Rb and selects it as learning data. For example, the data selection unit 13 makes two or more duplicates of the acceleration data in the time section Rb having a predetermined time width, and selects them as learning data. Note that the acceleration threshold Ab may be a value calculated from the acceleration at the determination time Lb, or may be a value set in advance. For example, the acceleration threshold Ab that is a value lower than the acceleration Ace that can be determined as “may be in an agitated state” and higher than the acceleration threshold A1 may be set, from analysis and a study result of past biological data of a plurality of persons.

In the above description, when the skill value of the nurse who made determination is “high skill”, the acceleration thresholds Aa and Ab are set and the time sections Ra and Rb having time widths before and after the determination time La and Lb are set. However, the method of setting the time sections Ra and Rb is not limited to that described above. For example, the data selection unit 13 may previously set a time width according to a label “being in an agitated state” or “may be in an agitated state”, and set the time sections Ra and Rb on the basis of the determination time La and Lb according to such a time width. At that time, the data selection unit 13 may set the time section in such a manner that the time width, that is, the time section, is changed according to the agitation level determined by the nurse, and in particular, that the time section is set to be longer as the agitation level is higher.

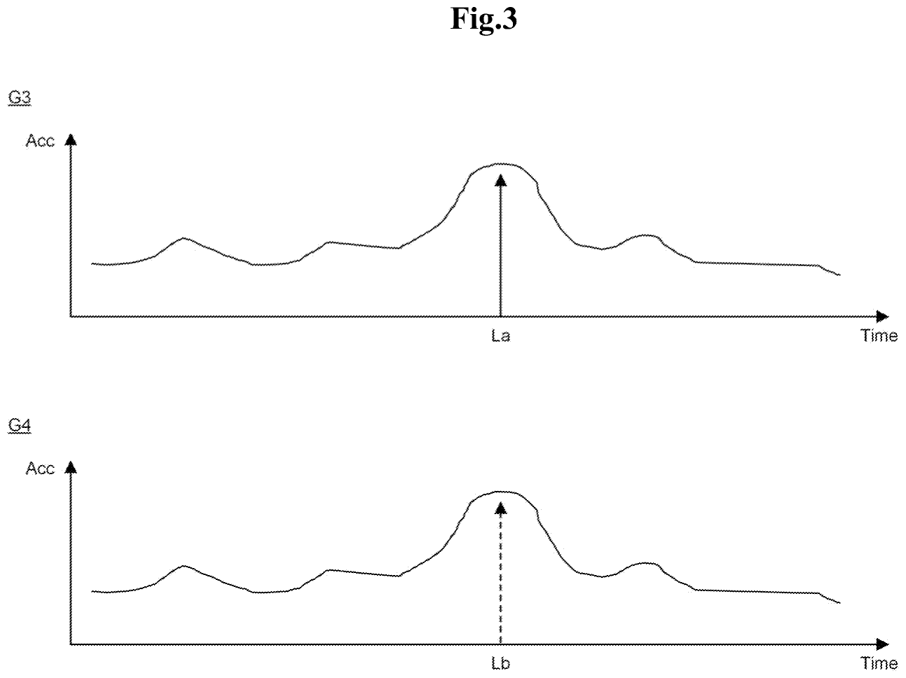

Next, an example of a data selection method in the case where the skill value of a nurse who made determination is “low skill” will be described with reference to FIG. 3. First, a reference sign G3 in the upper drawing of FIG. 3 indicates a method of selecting learning data from the acceleration Acc that is biological data to which a label “being in an agitated state” is given. In that case, the data selection unit 13 sets only biological data at the determination time to which a label “being in an agitated state” is given, as learning data. That is, when the skill value of a nurse is “low skill” and a label “being in an agitated state” is given, only acceleration data at the determination time La is selected as learning data, without selecting acceleration data in a time section having a long time width as described above. Moreover, in that case, the selected acceleration data is used as learning data without being duplicated.

A reference sign G4 in the lower drawing of FIG. 3 indicates a method of selecting learning data from the acceleration Acc that is biological data to which a label “may be in an agitated state” is given, in the case where the skill value of a nurse who made determination is “low skill”. In that case, even if there is biological data at the determination time Lb to which a label “may be in an agitated state” is given, the data selection unit 13 does not select such data as learning data. Therefore, in the case where the skill value of the nurse is low and the determined agitation level is low, learning data is not selected from biological data.

As described above, the data selection unit 13 selects acceleration data in a longer time section as the skill value of the nurse who made determination is higher, and duplicates it to use as learning data. Therefore, the data selection unit 13 selects a larger amount of learning data from biological data as the skill value of the nurse who made determination is higher. However, even in the case where the skill value of the nurse who made determination is “low skill”, it is possible to set a time section having a shorter time width than that in the case of “high skill” and select it as learning data, and duplicate it in the number smaller than that in the case of “high skill” and select it as learning data.

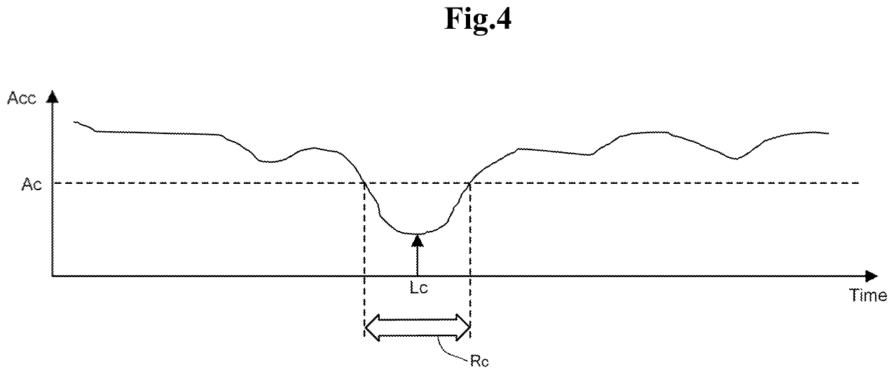

When it is determined that a patient “is in a calm state” by a nurse, the data selection unit 13 may select, from the biological data, learning data based on the determination time to which a label “being in a calm state” is given, and store it in the learning data storage unit 18 while giving a label “calm state”. Here, an example in which the skill value of a nurse who made determination is “high skill” and learning data is selected from biological data determined as “being in a calm state” will be described with reference to FIG. 4. The data selection unit 13 sets an acceleration threshold Ac that is a value higher than the acceleration Acc at determination time Lc to which a label “being in a calm state” is given, and sets a time section Re having a predetermined time width in which the acceleration Acc that is equal to or lower than the acceleration threshold Ac is measured. In this way, when the skill value of the nurse who made determination is “high skill”, the data selection unit 13 selects the acceleration Acc in the time section Re having the predetermined time width around the determination time Lc as learning data to which a label of calm state is given. Note that when the skill value of the nurse who made determination is “high skill”, the data selection unit 13 may make some duplicates of the acceleration data in the set time section Re and selects them as learning data. Further, when the skill value of the nurse who made determination is “low skill”, the data selection unit 13 may select only acceleration data at the determination time La as learning data, that is, without setting a time section having a time width, or may not select acceleration data at the determination time Lc as learning data.

The learning unit 14 reads, from the learning data storage unit 18, the learning data selected by the data selection unit 13 as described above, learns the acceleration data that is the learning data, and generates a model. Specifically, the learning unit 14 learns the acceleration data to which a label “agitated state” is given, that is, learning data, to thereby generate a model for detecting an agitated state from the acceleration data newly measured from a patient. Then, the learning unit 14 stores the generated model in the learning data storage unit 18. When there is acceleration data to which a label “calm state” is given in the learning data, the learning unit 14 may learn it to thereby generate a model for detecting each of an agitated state and a calm state from the acceleration data newly measured from a patient.

### Operation

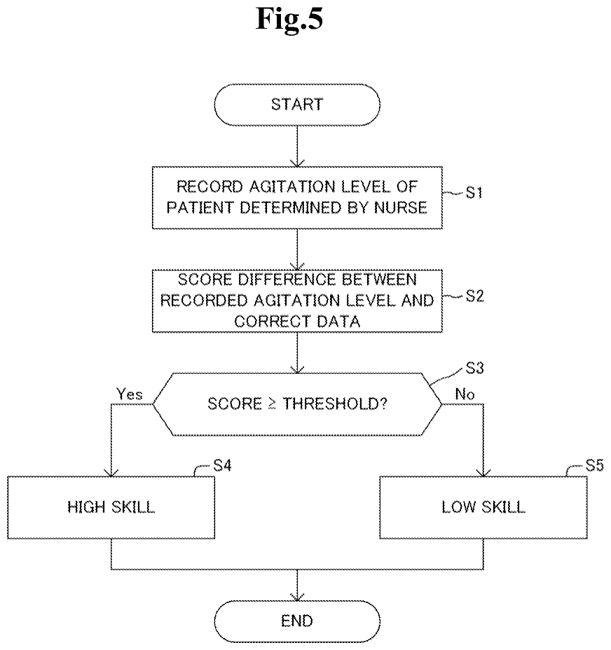

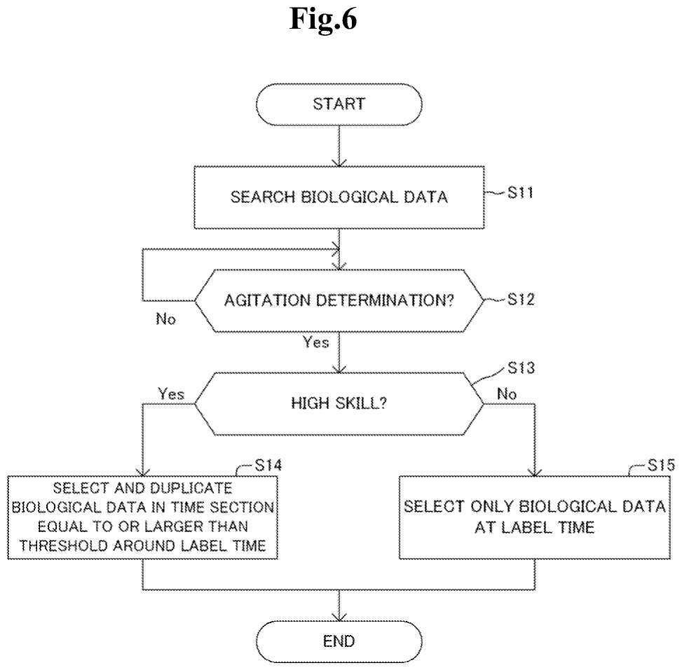

Next, operation of the information processing device 10 described above will be described with mainly reference to the flowcharts of FIGS. 5 and 6. First, an operation to determine a skill value of a nurse will be described with reference to the flowchart of FIG. 5.

First, the information processing device 10 stores therein biological data measured from a patient and imaging data in which motion of the patient is captured. Then, the information processing device 10 records a determination result of a state by a nurse with respect to the patient shown in the imaging data (step S1). In the present embodiment, as a determination result, the information processing device 10 records the agitation level such as “being in an agitated state” or “may be in an agitated state” determined by a nurse who reviews the imaging data of the patient, and the determination time.

Then, the information processing device 10 calculates a score according to a difference between a label indicating the agitation level that is a determination result by a nurse with respect to a patient, and a label that is correct data previously set with respect to the same patient (step S2). Then, the information processing device 10 checks whether the calculated value of the score is equal to or larger than a predetermined threshold (step S3), and when it is equal to or larger than the threshold (Yes at step S3), the information processing device 10 determines that the skill value of the nurse is high skill (step S4), while when it is less than the threshold, the information processing device 10 determines that the skill value of the nurse is low skill (step S5). Then, the information processing device 10 stores the skill value determined for each nurse in association with the identification information of the nurse.

Next, an operation to select learning data from biological data of a patient will be described with reference to the flowchart of FIG. 6.

First, the information processing device 10 searches the biological data of a patient for the determination time at which it is determined as “being in an agitated state” or “may be in an agitated state”, that is, the determination time to which an agitation label is given (step S11). Then, when there is determination time to which an agitation label is given (Yes at step S12), the information processing device 10 selects learning data from the biological data on the basis of the determination time to which a label of agitation is given. At that time, the information processing device 10 first checks the skill value of a nurse who made determination (step S13). Then, when the skill value of the nurse is “high skill” (Yes at step S13), the information processing device 10 sets the time sections Ra and Rb having a predetermined time width around the determination time La and Lb as illustrated in FIG. 2, duplicates the biological data in the time sections Ra and Rb, and selects them as learning data (step S14). Then, the information processing device 10 stores the biological data selected as learning data while giving a “agitated state” label thereto. At that time, the information processing device 10 may change the lengths of the time sections Ra and Rb to select the biological data according to the level of determination of an agitated state by the nurse, like a difference between the reference sign G1 and the reference sing G2 in FIG. 2.

Further, when the skill value of the nurse is “low skill” (No at step S13), the information processing device 10 sets only biological data at the determination time La as learning data, as illustrated in the upper drawing of FIG. 3 (step S15). However, when a label “being in an agitated state” is given as illustrated in the lower drawing of FIG. 3, the information processing device 10 may not select biological data at the determination time Lb as learning data.

In this way, the information processing device 10 selects acceleration data in a longer time section as the skill value of the nurse who made determination is higher, and duplicates it to use as learning data. Therefore, the information processing device 10 selects a larger amount of learning data from biological data as the skill value of the nurse who made determination is higher.

Then, the information processing device 10 learns the acceleration data that is learning data selected as described above, and generates a model for detecting an agitated state. Further, the information processing device 10 uses the generated model to detect an agitated state from biological data newly measured from a patient.

As described above, in the present embodiment, as the skill value that is ability to determine an agitated state by a nurse is higher, a larger amount of biological data of a patient determined to be in an agitated state by the nurse is selected as learning data. Therefore, it is possible to improve the quality of biological data to be learned. Further, by learning high-quality biological data and generating a model for detecting an agitated state, it is possible to improve the accuracy of detecting an agitated state using such a model.

### Second Example Embodiment

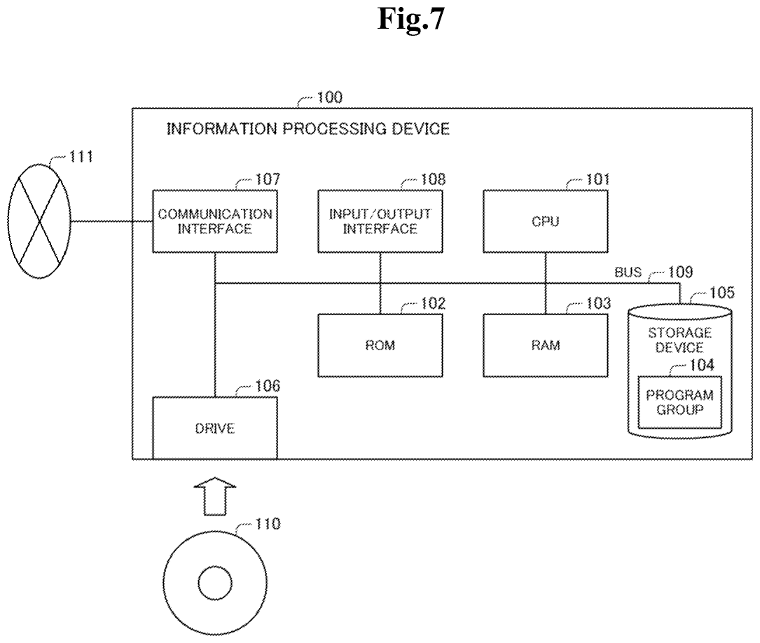

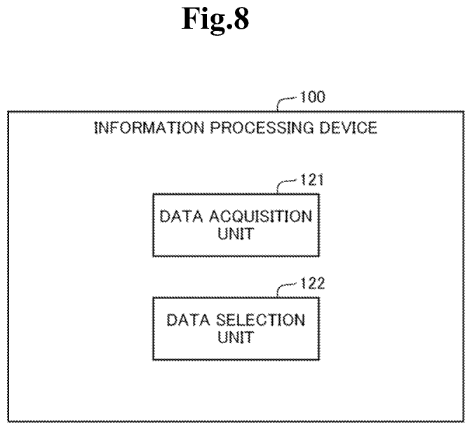

Next, a second example embodiment of the present disclosure will be described with reference to FIGS. 7 and 8. FIGS. 7 and 8 are block diagrams illustrating a configuration of an information processing device according to the second example embodiment. Note that the present embodiment shows the outline of the configuration of the information processing device explained in the above-described embodiment.

First, a hardware configuration of an information processing device 100 in the present embodiment will be described with reference to FIG. 7. The information processing device 100 is configured of a typical information processing device (computer), having a hardware configuration as described below as an example.
Central Processing Unit (CPU) 101 (arithmetic unit)
Read Only Memory (ROM) 102 (storage device)
Random Access Memory (RAM) 103 (storage device)
Program group 104 to be loaded to the RAM 103
Storage device 105 storing therein the program group 104
Drive 106 that performs reading and writing on a storage medium 110 outside the information processing device
Communication interface 107 connecting to a communication network 111 outside the information processing device
Input/output interface 108 for performing input/output of data
Bus 109 connecting the respective constituent elements

Note that FIG. 7 illustrates an example of a hardware configuration of an information processing device that is the information processing device 100. The hardware configuration of the information processing device is not limited to that described above. For example, the information processing device may be configured of part of the configuration described above, such as without the drive 106. Moreover, instead of the CPU, the information processing device may use a Graphic Processing Unit (GPU), a Digital Signal Processor (DSP), a Micro Processing Unit (MPU), a Floating Point number processing Unit (FPU), a Physics Processing Unit (PPU), a Tensor Processing Unit (TPU), a quantum processor, a microcontroller, or a combination thereof.

The information processing device 100 can construct, and can be equipped with, a data acquisition unit 121 and a data selection unit 122 illustrated in FIG. 8 through acquisition and execution of the program group 104 by the CPU 101. Note that the program group 104 is stored in the storage device 105 or the ROM 102 in advance, and is loaded to the RAM 103 and executed by the CPU 101 as needed. Further, the program group 104 may be provided to the CPU 101 via the communication network 111, or may be stored on the storage medium 110 in advance and read out by the drive 106 and supplied to the CPU 101. However, the data acquisition unit 121 and the data selection unit 122 may be constructed by dedicated electronic circuits for implementing such means.

The data acquisition unit 121 acquires biological data measured from a person whose state related to agitation is to be determined, and a determination result of the state by a determiner with respect to the person. For example, the data acquisition unit 121 acquires a determination result of an agitated state of a person.

The data selection unit 122 selects learning data from the biological data, on the basis of a skill value representing the ability of determining the state set for the determiner, and the determination result. For example, as the skill value is higher, the data selection unit 122 selects a larger amount of learning data from the biological data determined as being in an agitated state.

Since the present disclosure is configured as described above, as the skill value of a determiner is higher, a larger amount of biological data of a person determined by such a determiner is selected as learning data. Therefore, it is possible to improve the quality of biological data to be learned. Further, by learning high-quality biological data and generating a model for detecting a state, it is possible to improve the accuracy of detecting a state using such a model.

Note that the program described above can be stored in a non-transitory computer-readable medium of any type and supplied to a computer. Non-transitory computer-readable media include tangible storage media of various types. Examples of non-transitory computer-readable media include magnetic storage media (for example, flexible disk, magnetic tape, and hard disk drive), magneto-optical storage media (for example, magneto-optical disk), a CD-ROM (Read Only Memory), a CD-R, a CD-R/W, and semiconductor memories (for example, mask ROM, PROM (Programmable ROM), EPROM (Erasable PROM), flash ROM, and RAM (Random Access Memory)). The program may be supplied to a computer by a transitory computer-readable medium of any type. Examples of transitory computer-readable media include electric signals, optical signals, and electromagnetic waves. A transitory computer-readable medium can supply the program to a computer via a wired communication channel such as a wire and an optical fiber, or a wireless communication channel.

While the present disclosure has been described with reference to the example embodiments described above, the present disclosure is not limited to the above-described embodiments. The form and details of the present disclosure can be changed within the scope of the present disclosure in various manners that can be understood by those skilled in the art. Further, at least one of the functions of the data acquisition unit 121 and the data selection unit 122 described above may be carried out by an information processing device provided and connected to any location on the network, that is, may be carried out by so-called cloud computing.

### Supplementary Notes

The whole or part of the example embodiments disclosed above can be described as the following supplementary notes. Hereinafter, outlines of the configurations of an information processing device, an information processing method, and a program, according to the present disclosure, will be described. However, the present disclosure is not limited to the configurations described below.

### (Supplementary Note 1)

An information processing device comprising:
a data acquisition unit that acquires biological data measured from a person whose state related to agitation is to be determined, and a determination result of the state by a determiner with respect to the person; and
a data selection unit that selects learning data from the biological data on a basis of a skill value and the determination result, the skill value representing ability of determining the state and being set for the determiner.

### (Supplementary Note 2)

The information processing device according to supplementary note 1, wherein
the data selection unit selects the learning data from the biological data by means of a selection method previously set according to the skill value of the determiner.

### (Supplementary Note 3)

The information processing device according to supplementary note 1 or 2, wherein
the data selection unit selects a larger amount of learning data from the biological data as the skill value of the determiner is higher.

### (Supplementary Note 4)

The information processing device according to any of supplementary notes 1 to 3, wherein
the data selection unit selects the learning data by duplicating the biological data as the skill value of the determiner is higher.

### (Supplementary Note 5)

The information processing device according to any of supplementary notes 1 to 4, wherein
the data acquisition unit acquires the biological data that is time-series data measured from the person, and the determination result including a determination time of the state by the determiner with respect to the person, and
the data selection unit sets a time section of the biological data on a basis of the determination time according to the skill value of the determiner, and selects the biological data in the set time section as the learning data.

### (Supplementary Note 6)

The information processing device according to supplementary note 5, wherein
the data selection unit sets the time section longer as the skill value of the determiner is higher.

### (Supplementary Note 7)

The information processing device according to supplementary note 5 or 6, wherein
the data acquisition unit acquires the determination result including a level of the state determined by the determiner, and
the data selection unit sets a time section of the biological data on the basis of the determination time according to the level of the state, and selects the biological data in the set time section as the learning data.

### (Supplementary Note 8)

The information processing device according to any of supplementary notes 1 to 7, further comprising
a skill determination unit that determines the skill value of the determiner on a basis of the determination result by the determiner with respect to the person and correct data of the state for the person.

### (Supplementary Note 9)

An information processing method comprising:
acquiring biological data measured from a person whose state related to agitation is to be determined, and a determination result of the state by a determiner with respect to the person; and
selecting learning data from the biological data on a basis of a skill value and the determination result, the skill value representing ability of determining the state and being set for the determiner.

### (Supplementary Note 9.1)

The information processing method according to supplementary note 9, further comprising
selecting the learning data from the biological data by means of a selection method previously set according to the skill value of the determiner.

### (Supplementary Note 9.2)

The information processing method according to supplementary note 9 or 9.1, wherein
the data selection unit selects a larger amount of learning data from the biological data as the skill value of the determiner is higher.

### (Supplementary Note 9.3)

The information processing method according to any of supplementary notes 9 to 9.2, wherein
the data selection unit selects the learning data by duplicating the biological data as the skill value of the determiner is higher.

### (Supplementary Note 9.4)

The information processing method according to any of supplementary notes 9 to 9.3, wherein
the data acquisition unit acquires the biological data that is time-series data measured from the person, and the determination result including a determination time of the state by the determiner with respect to the person, and
the data selection unit sets a time section of the biological data on a basis of the determination time according to the skill value of the determiner, and selects the biological data in the set time section as the learning data.

### (Supplementary Note 9.5)

The information processing method according to supplementary note 9.4, wherein
the data selection unit sets the time section longer as the skill value of the determiner is higher.

### (Supplementary Note 9.6)

The information processing method according to supplementary note 9.4 or 9.5, wherein
the data acquisition unit acquires the determination result including a level of the state determined by the determiner, and
the data selection unit sets a time section of the biological data on the basis of the determination time according to the level of the state, and selects the biological data in the set time section as the learning data.

### (Supplementary Note 9.7)

The information processing method according to any of supplementary notes 9 to 9.6, further comprising
a skill determination unit that determines the skill value of the determiner on a basis of the determination result by the determiner with respect to the person and correct data of the state for the person.

### (Supplementary Note 10)

A program for causing a computer to execute processing to:
acquire biological data measured from a person whose state related to agitation is to be determined, and a determination result of the state by a determiner with respect to the person; and
select learning data from the biological data on a basis of a skill value and the determination result, the skill value representing ability of determining the state and being set for the determiner.

### REFERENCE SIGNS LIST

10 information processing device
11 data acquisition unit
12 skill determination unit
13 data selection unit
14 learning unit
16 patient data storage unit
17 nurse data storage unit
18 learning data storage unit
100 information processing device
101 CPU
102 ROM
103 RAM
104 program group
105 storage device
106 drive
107 communication interface
108 input/output interface
109 bus
110 storage medium
111 communication network
121 data acquisition unit
122 data selection unit

What is claimed is:

1. An information processing device comprising:
at least one memory configured to store instructions; and
at least one processor configured to execute instructions to:
acquire biological data measured from a person whose state related to agitation is to be determined, and a determination result of the state by a determiner with respect to the person; and
select learning data to be used for performing machine learning from the biological data on a basis of a skill value and the determination result, the skill value representing ability of determining the state and being set for the determiner.

2. The information processing device according to claim 1, wherein the at least one processor is configured to execute the instructions to
select the learning data from the biological data by means of a selection method previously set according to the skill value of the determiner.

3. The information processing device according to claim 1, wherein the at least one processor is configured to execute the instructions to
select a larger amount of learning data from the biological data as the skill value of the determiner is higher.

4. The information processing device according to claim 1, wherein the at least one processor is configured to execute the instructions to
select the learning data by duplicating the biological data as the skill value of the determiner is higher.

5. The information processing device according to claim 1, wherein the at least one processor is configured to execute the instructions to:
acquire the biological data that is time-series data measured from the person, and the determination result including a determination time of the state by the determiner with respect to the person; and
set a time section of the biological data on a basis of the determination time according to the skill value of the determiner, and select the biological data in the set time section as the learning data.

6. The information processing device according to claim 5, wherein the at least one processor is configured to execute the instructions to
set the time section longer as the skill value of the determiner is higher.

7. The information processing device according to claim 5, wherein the at least one processor is configured to execute the instructions to:
acquire the determination result including a level of the state determined by the determiner; and
set a time section of the biological data on the basis of the determination time according to the level of the state, and select the biological data in the set time section as the learning data.

8. The information processing device according to claim 1, wherein the at least one processor is configured to execute the instructions to
determine the skill value of the determiner on a basis of the determination result by the determiner with respect to the person and correct data of the state for the person.

9. An information processing method comprising:
acquiring biological data measured from a person whose state related to agitation is to be determined, and a determination result of the state by a determiner with respect to the person; and
selecting learning data from the biological data on a basis of a skill value and the determination result, the skill value representing ability of determining the state and being set for the determiner.

10. A non-transitory computer-readable medium storing thereon a program comprising instructions for causing a computer to execute processing to:
acquire biological data measured from a person whose state related to agitation is to be determined, and a determination result of the state by a determiner with respect to the person; and
select learning data from the biological data on a basis of a skill value and the determination result, the skill value representing ability of determining the state and being set for the determiner.
